(19) United States
(12) Patent Application Publication
(10) Pub. No.: US 2007/0073352 A1
(43) Pub. Date: Mar. 29, 2007

(54) Method and apparatus for regulating a cardiac stimulation therapy

(76) Inventors: David E. Euler, Maple Grove, MN (US); Tommy D. Bennett, Shoreview, MN (US); Ven Manda, Stillwater, MN (US); D. Curtis Deno, Andover, MN (US); Vincent E. Splett, Apple Valley, MN (US)

Correspondence Address: MEDTRONIC, INC., 710 MEDTRONIC PARK, MINNEAPOLIS, MN, 55432-9924, US

(21) Appl. No.: 11/237,177

(22) Filed: Sep. 28, 2005

(51) Int. Cl. A61N 1/00

(52) U.S. Cl. 607/23

(57) ABSTRACT

A method comprising sensing a blood pressure signal, deriving a hemodynamic measure from the sensed blood pressure signal, adjusting an extra systolic stimulation control parameter in response to the hemodynamic measure, and delivering extra systolic stimulation pulses according to the adjusted control parameter. The sensed blood pressure signal may be a ventricular or arterial blood pressure signal from which an estimated cardiac output, end diastolic pressure, mean pressure or any other hemodynamic measure is derived. Adjusting the extra systolic stimulation control parameter may include adjusting a pacing rate, a pacing interval, an extra systolic stimulation ratio, an extra systolic stimulation interval or enabling or terminating the extra systolic stimulation.

### TECHNICAL FIELD

The invention relates to implantable cardiac stimulation devices, and, more particularly, to a method for regulating the delivery of cardiac stimulation pulses.

### BACKGROUND

Post-extra systolic potentiation (PESP) is a property of cardiac myocytes that results in enhanced mechanical function of the heart on the beats following an extra systolic stimulus delivered early after either an intrinsic or pacing-induced systole. The magnitude of the enhanced mechanical function is strongly dependent on the timing of the extra systole relative to the preceding intrinsic or paced systole. When correctly timed, an extra systolic stimulation pulse causes an electrical depolarization of the heart but the attendant mechanical contraction is absent or substantially weakened. The contractility of the subsequent cardiac cycles, referred to as the post-extra systolic beats, is increased. This phenomenon is also described in detail in commonly assigned U.S. Pat. No. 5,213,098 issued to Bennett et al., incorporated herein by reference in its entirety.

The mechanism of PESP is thought to involve the calcium cycling within the myocytes. The extra systole initiates a limited calcium release from the sarcoplasmic reticulum (SR). The limited amount of calcium that is released in response to the extra systole is not enough to cause a normal mechanical contraction of the heart. After the extra systole, the SR continues to take up calcium with the result that subsequent depolarization(s) cause a larger release of calcium from the SR, resulting in an increase in the strength of myocyte contraction and an increase in stroke volume from the cardiac chamber.

As noted, the degree of mechanical augmentation on post-extra systolic beats depends strongly on the time interval between a primary systole and the subsequent extra systole, referred to herein as the “extra systolic interval” (ESI). If the ESI is too long, the PESP effects are not achieved because a normal mechanical contraction takes place in response to the extra systolic stimulus. As the ESI is shortened, a maximal effect is reached when the ESI is slightly longer than the myocardial refractory period. At this ESI, an electrical depolarization occurs without a mechanical contraction or with a substantially weakened contraction. When the ESI becomes too short, the stimulus falls within the absolute refractory period and there is no depolarization or contraction and PESP does not occur.

The effects of PESP may advantageously benefit patients suffering from cardiac mechanical insufficiency, such as patients in heart failure. Extra systolic stimulation (ESS) can be delivered by paired pacing, an extra systolic stimulus delivered after a primary pacing pulse, or coupled pacing, an extra systolic stimulus delivered after an intrinsic heart beat. Both can enhance mechanical cardiac function for one or more beats following the extra systolic stimulus. Another effect of ESS is a slowing of the mechanical heart rate. The mechanical heart rate slows because the extra systolic beats are too weak to eject blood from the ventricles and in this state the mechanical heart rate (i.e., the arterial pulse rate) is less than the electrical heart rate. A decrease in the mechanical heart rate, however, may not be beneficial in all patients, particularly if the slowed heart rate results in an unacceptable decrease in cardiac output. In order to realize the benefits of ESS in patients having mechanical dysfunction, methods and associated apparatus for regulating ESS are needed.

### BRIEF DESCRIPTION OF THE DRAWINGS

FIG. 1 is a conceptual overview of a system according to one embodiment of the invention.

FIG. 2 depicts a system architecture of an illustrative embodiment of the dual chamber cardiac stimulation device shown in FIG. 1.

FIG. 3A illustrates the delivery of dual chamber ESS therapy.

FIG. 3B illustrates ESS control parameters that may be used in a bi-ventricular ESS application.

FIG. 3C illustrates ESS control parameters that may be used in a bi-atrial ESS application.

FIG. 4 is a flow chart summarizing a general method for regulating ESS based on hemodynamic monitoring.

FIG. 5 is a flow chart summarizing one embodiment for regulating ESS that includes adjusting a pacing rate in response to hemodynamic monitoring.

FIG. 6 is a flow chart summarizing one embodiment of a method for regulating ESS that includes adjusting the ESS ratio in response to a hemodynamic measure.

FIG. 7 is a flow chart summarizing another embodiment of a method for regulating ESS that includes adjusting an ESI in response to a hemodynamic measure.

FIG. 8 shows a right ventricular pressure (RVP) waveform and a pulmonary artery pressure (PAP) waveform and illustrates a number of hemodynamic measures that may be derived from a pressure signal for use in regulating ESS.

### DETAILED DESCRIPTION

In the following description, references are made to illustrative embodiments for carrying out the invention. It is understood that other embodiments may be utilized without departing from the scope of the invention.

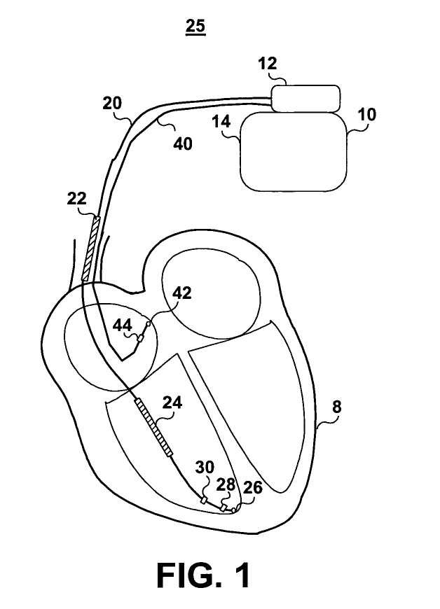

FIG. 1 is a conceptual overview of a system according to one embodiment of the invention. FIG. 1 illustrates a system 25 including a cardiac stimulation device 10 connected to a one or more cardiac leads 20 and 40 deployed in a patient's heart 8 for physiological monitoring and for delivering a stimulation therapy. Cardiac stimulation device 10 collects and processes data about heart 8 from one or more sensors, including a pressure sensor 30 and any of electrodes 22, 24, 26, 28, 42 and 44 for sensing cardiac electrogram (EGM) signals. Cardiac stimulation device 10 provides a therapy or other response to the patient as appropriate, and as described more fully below. In particular, cardiac stimulation device 10 delivers ESS therapy, which is controlled by device 10, at least in part, in response to blood pressure signals received from blood pressure sensor 30.

Cardiac stimulation device 10 is provided with a hermetically-sealed housing 14 that encloses a processor, memory, and other components as appropriate to produce the desired functionalities of the device 10. Device 10 includes a connector header 12 for receiving leads 20 and 40 and facilitating electrical connection of leads 20 and 40 to the components enclosed in housing 14. In various embodiments, cardiac stimulation device 10 is implemented as any implantable medical device capable of measuring the heart rate of a patient and a pressure signal and is further capable of delivering ESS pulses. Device 10 may additionally include other monitoring capabilities, such as, but not limited to, lung wetness monitoring, heart wall motion monitoring, blood chemistry monitoring or other physiological monitoring. Device 10 may further include other therapy delivery capabilities such as, but not limited to, any type of cardiac pacing therapy, cardioversion, defibrillation, drug delivery, or neurostimulation. Examples of a suitable device that may be used in various embodiments of the invention is generally described in commonly assigned U.S. Pat. No. 6,438,408B1 issued to Mulligan et al., and in U.S. Pat. No. 6,738,667B2 issued to Deno et al., both of which patents are incorporated herein by reference in their entirety. An example of an implantable device capable of measuring right ventricular pressure is the CHRONICLE® monitoring device available from Medtronic, Inc. of Minneapolis, Minn., which includes a mechanical sensor capable of detecting a ventricular pressure signal.

In the example of FIG. 1, cardiac stimulation device 10 receives a right ventricular endocardial lead 20 and a right atrial endocardial lead 40, although the particular cardiac leads used may vary from embodiment to embodiment. Ventricular lead 20 is provided with a tip electrode 26 and ring electrode 28 for sensing ventricular EGM signals and for delivering cardiac stimulation pulses in the ventricle. Ventricular lead 20 is also shown having defibrillation coil electrodes 22 and 24 in the event cardiac stimulation device 10 is configured to provide cardioversion and/or defibrillation therapies. Atrial lead 40 is provided with a tip electrode 42 and ring electrode 44 for sensing atrial EGM signals and for delivering cardiac stimulation pulses in the atrium. Atrial lead 40 and ventricular lead 20 can be used to deliver pacing stimuli in a coordinated fashion to provide dual chamber pacing and are used to deliver ESS pulses following either sensed, intrinsic cardiac events or paced events. In addition, the stimulation device housing 14 may function as an electrode, along with other electrodes that may be provided at various locations on the housing of device 10. In alternate embodiments, other data inputs, leads, electrodes and the like may be provided.

The cardiac stimulation device 10 shown in FIG. 1 is a dual chamber device capable of sensing and stimulating in an atrial and ventricular chamber. However, it is understood that in various embodiments of the invention the illustrative device 10 of FIG. 1 could be programmably or physically modified to function as a single chamber or multi-chamber system for monitoring and/or stimulating in one or more heart chambers.

In operation, cardiac stimulation device 10 obtains data about heart 8 via leads 20 and 40 and/or other sources. This data is provided to a processor enclosed in housing 14, which suitably analyzes the data, stores appropriate data in associated memory, and/or provides a response as appropriate. In particular, cardiac stimulation device 10 selects or adjusts a therapy and regulates the delivery of the therapy. Specifically, as will be described in greater detail below, cardiac stimulation device 10 obtains pressure data input from pressure sensor 30 that is carried by right ventricular endocardial lead 20. In other embodiments, pressure sensor 30 may be carried by a separate lead. For example, in some embodiments, cardiac stimulation device 10 may be provided having electrodes for sensing and stimulation functions carried on subcutaneous leads or built into the housing 14 of device 10 and not require electrodes carried by endocardial leads as shown in FIG. 1. The pressure data obtained from sensor 30 is used by control circuitry included in device 10 for regulating the delivery of ESS pulses. Pressure sensor 30 is shown in FIG. 1 deployed in the right ventricle for measuring right ventricular pressure. In alternative embodiments, pressure sensor 30 may be positioned appropriately for generating a signal responsive to left ventricular pressure changes, arterial pressure changes or atrial pressure changes. As such, a lead system provided for use with device 10 may include a coronary sinus lead or other lead that allows left atrial and/or left ventricular pressure signals to be captured, and/or leads having a pressure sensor disposed for sensing arterial pressure signals.

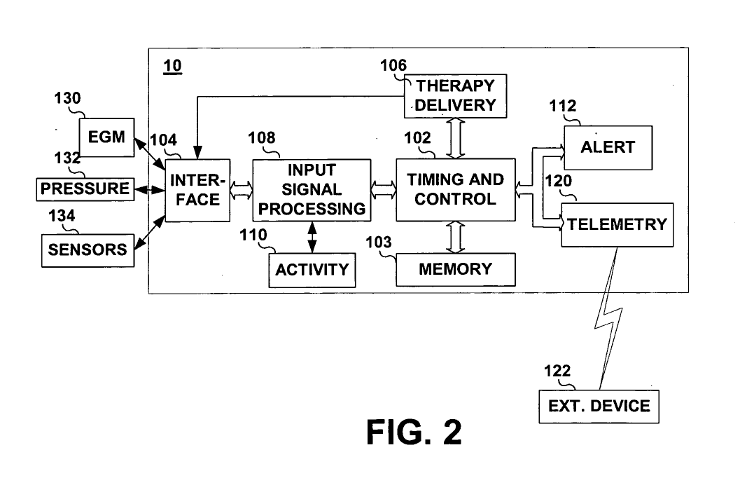

FIG. 2 depicts a system architecture of an illustrative embodiment of a dual chamber cardiac stimulation device 10. The system architecture is typically constructed about a micro-processor based control and timing module 102 which varies in sophistication and complexity depending upon the type and functional features incorporated therein. Timing and control module 102 may be implemented with any type of microprocessor, digital signal processor, application specific integrated circuit (ASIC), field programmable gate array (FPGA), state machine circuitry, or other integrated or discrete logic circuitry programmed or otherwise configured to provide functionality as described herein. Timing and control module 102 executes instructions stored in digital memory 103 to provide functionality as described below. Instructions provided to timing and control module 102 may be executed in any manner, using any data structures, architecture, programming language and/or other techniques. Digital memory 103 is any storage medium capable of maintaining digital data and instructions provided to timing and control module 102 such as a static or dynamic random access memory (RAM), or any other electronic, magnetic, optical or other storage medium.

Cardiac stimulation device 10 includes interface 104 for interfacing circuitry included in device 10 with the various electrodes and sensors deployed to operating sites within the patient's body. Interface 104 allows therapy delivery module 106 to be coupled to selected electrodes for delivering cardiac stimulation pulses. In particular, therapy delivery module 106 delivers cardiac pacing pulses and ESS pulses as regulated by timing and control 102. Therapy delivery module 106 may further deliver cardioversion and defibrillation pulses or other cardiac stimulation therapies. Other therapies may be included in therapy delivery module 106 such as a drug delivery pump.

Interface 104 also provides signals received from sensing electrodes and a blood pressure sensor (as shown in FIG. 1), and any other physiological sensor to input signal processing module 108. Input signal processing module 108 uses EGM signals 130 received from sensing electrodes and blood pressure signals 132 from the pressure sensor to compute one or more hemodynamic parameters, along with determining a heart rate, which are used in regulating the delivery of ESS therapy. Interface 104 may further receive other sensor signals 134 in various embodiments.

Input signal processing module 108 includes at least one sense amplifier circuit for receiving cardiac EGM signals 130 for use in sensing cardiac events. Such signals used by timing and control module 102 in controlling and adjusting therapies delivered by therapy delivery module 104. With regard to the dual chamber device illustrated in FIG. 1, signal processing module 108 includes atrial and ventricular sense amplifier channels for sensing atrial events and ventricular events and determining a heart rate and detecting the heart rhythm. Accordingly, timing and control module 102 responds by adjusting the delivery of ESS pulses as appropriate and/or any other therapies delivered by therapy delivery module 104.

In addition, input signal processing module 108 includes at least one physiologic sensor signal processing channel for sensing and processing at least a blood pressure signal. In the embodiment shown in FIG. 1, a signal processing channel is provided for processing a right ventricular blood pressure signal. As will be described herein, the blood pressure signal is used to derive a hemodynamic parameter used for regulating ESS therapy. In a particular embodiment, an estimate of cardiac output derived from a right ventricular pressure signal is used in regulating ESS therapy. In other embodiments, an arterial, left ventricular, right atrial or left atrial pressure signal may be used for estimating cardiac output.

Monitoring of signals received by input signal processor 108 may be performed continuously or discontinuously, on a periodic or triggered basis. Physiological data and/or device related data may be stored continuously or triggered upon a physiological event or a manual trigger. Uplink and downlink telemetry capabilities are provided by telemetry circuit 120 to enable communication with an external medical device 122, which may be a home monitor or a programmer. Stored physiologic and/or device-related data can be transferred to the external medical device 122 and may be further transmitted to a remote patient management center via an appropriate communications network.

Device 10 may further include an activity sensor 110 for deriving the level of a patient's activity. The implementation of activity sensors in cardiac pacemaking devices is known in the art. Activity sensor 110 may further include a posture sensor for indicating the position of the patient. A posture sensor signal can be used, either alone or in combination with an activity sensor signal for determining or confirming a resting or active state of the patient. An activity and/or posture signal may be used in controlling the ESS therapy.

In some embodiments, device 10 includes a patient alert 112 for notifying the patient of a particular physiological or device-related event Patient notification is provided by perceivable sensory stimulation, which may be an audible tone, vibration, muscle stimulation or the like. For example, the patient alert 112 may notify a patient of a hemodynamic event that warrants medical attention.

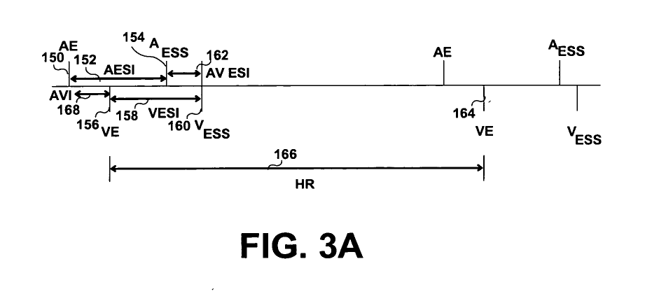

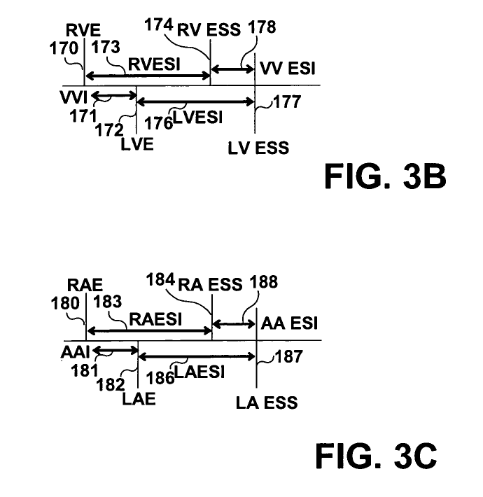

FIG. 3A illustrates the delivery of dual chamber ESS therapy. Dual chamber ESS can be delivered during either normal sinus rhythm or during cardiac pacing. An atrial event (AE) 150, which may be either an atrial paced event or an intrinsic atrial sensed event, is followed by an atrial extra systolic interval (AESI) 152 and the delivery of an atrial ESS pulse (A_{ESS}) 154. A ventricular event (VE) 156 is similarly followed by a ventricular extra systolic interval (VESI) 158 and the delivery of a ventricular ESS pulse (V_{ESS}) 160. The time interval between the atrial ESS pulse 154 and the ventricular ESS pulse 160 is referred to as the AV extra systolic interval (AVESI) 162.

When the AE 150 and the VE 156 are intrinsic events, delivery of ESS pulses is referred to as “coupled pacing.” When the AE 150 and the VE 156 are paced events, delivery of the ESS pulses is referred to as “paired pacing.” At times, the AE 150 may be a paced event and the VE 156 may be an intrinsic event conducted from the atria. At other times, the AE 150 may be a sensed event and the VE 156 may be a paced event following AE 150, for example in patients having AV block. As such “coupled pacing” may be occurring in one chamber while “paired pacing” may be occurring in another chamber. Separate atrial ESIs and ventricular ESIs may be defined for both paired pacing and coupled pacing situations. Since post-extra systolic potentiation occurs in both atrial and ventricular myocytes, separate adjustment of the atrial and ventricular ESIs may be necessary to achieve optimal hemodynamic performance. As referred to herein, “ESS” refers to either coupled or paired pacing or a combination of both in dual or multi-chamber ESS applications.

The mechanical heart rate (HR) 166 is determined by the rate of the primary ventricular or atrial events, which may be an intrinsic or paced rate. Since a mechanical response to the ESS pulse is absent or substantially weakened, the electrical rate will be higher than the mechanical rate during ESS therapy.

ESS pulses may be delivered on each cardiac cycle, i.e., at a 1:1 ratio with the cardiac paced or intrinsic rate. The electrical rate would be double the mechanical rate. ESS pulses may alternatively be delivered at a rate less than the heart rate, e.g., every other cardiac cycle or at a 2:1 ratio with the paced or intrinsic rate, every third cardiac cycle or at a 3:1 ratio with the paced or intrinsic rate, and so on. The ratio of paced or intrinsic events to ESS pulses is one parameter that can be regulated in response to a hemodynamic measure derived from a blood pressure signal.

Other ESS control parameters that can be regulated in response to a hemodynamic measure derived from a blood pressure signal include the atrial ESI 152 and the ventricular ESI 158. In some embodiments, the timing of the atrial ESS pulse 154 may be controlled by the AV ESI 162. After the primary VE 156, a VESI 158 is set and the AESS pulse 154 is delivered an interval equal to the AV ESI 162 prior to the scheduled VESS pulse 160. The AV ESI 162 may be adjusted in response to a hemodynamic measure derived from a blood pressure signal. Adjustments of the various ESIs will affect the magnitude of the mechanical responses in both the atria and ventricles to the ESS pulses and therefore the degree of post-extra systolic potentiation occurring on the subsequent heart beat.

The HR 166 is expected to decrease in response to ESS. In some patients, a decrease in HR may offset the increase in stroke volume that occurs on potentiated beats resulting in an overall decrease in cardiac output (CO). As such, the HR 166 may be controlled during ESS therapy by controlling the atrial pacing rate. The atrial pacing rate is thus another ESS control parameter than can be regulated in response to a hemodynamic measure, in particular an estimated CO, derived from a blood pressure signal. As will be described in greater detail below, a decrease in CO can be responded to by setting an atrial pacing rate greater than the intrinsic heart rate.

If ventricular pacing is necessary, for example in patients having AV block, the ventricular pacing rate may track the atrial pacing rate. Ventricular pacing pulses are delivered at an A-V interval (AVI) 168. The AVI 168 may be adjusted to control the timing of VE 156. AVI 168 may be adjusted in response to a hemodynamic measure derived from a blood pressure signal during ESS therapy. Ventricular pacing may also be delivered to regulate the ventricular rate independent of the atrial rate, for example in patients having sustained or intermittent atrial tachycardia. As such the ventricular pacing rate may be an ESS control parameter that is adjusted in response to a hemodynamic measure derived from a blood pressure signal.

While a dual chamber ESS application is illustrated in FIG. 3A, it is recognized that the various ESS control parameters described can be simplified or expanded for single chamber, bi-ventricular, or multi-chamber ESS therapy applications. FIG. 3B illustrates ESS control parameters that may be used in a multi-chamber or bi-ventricular ESS application. A right ventricular (RV) ESI 173 is used to control the timing of a RV ESS pulse 174 following a RV event 170. A left ventricular (LV) ESI 176 is used to control the timing of a LV ESS pulse 177 following a LV event 172. RV event 170 and LV event 172 may be intrinsic depolarizations or one or both may be paced events separated by a VV interval 171. A V-V ESI 178 may exist relating to the time interval between the RV ESS pulse 174 and the LV ESS pulse 177. The V-V interval controlling ventricular synchronization of the primary ventricular events RVE 170 and LVE 172, and any of the ESIs 173, 176 and/or 178 controlling the timing of left and right ventricular ESS pulses 174 and 177 are considered ESS control parameters that can be adjusted in response to a hemodynamic measure derived from a blood pressure signal.

Likewise, as shown in FIG. 3C, during a bi-atrial ESS therapy application or a multi-chamber application that involves both atria, a right atrial (RA) ESI 183 may be used to control the timing of a RA ESS pulse 184 following a RA event 180. A left atrial (LA) ESI 186 may be used to control the timing of a LA ESS pulse 187 following a LA event 182. In some embodiments, an A-A ESI 188 is used to control the time interval between a RA ESS pulse 184 and left atrial (LA) ESS pulse 187. The RA event 180 and LA event 182 may be intrinsic or paced events. The atrial pacing rate as well as the A-A interval 181 controlling the timing between RA event 180 and LA event 182 during pacing of either or both atrial chambers may be adjusted in response to a hemodynamic measure derived from a blood pressure signal.

In summary, in any single, dual or multi-chamber mode, control parameters for regulating an ESS therapy include, but are not limited to, a pacing rate, a pacing interval between two cardiac chambers (AV interval, AA interval or VV interval), the ESS ratio of primary cardiac events (paced or sensed) to ESS events, and any ESI used to control the timing of ESS pulses relative to a primary atrial or ventricular event or another ESS pulse.

The timing diagrams shown in FIGS. 3A, 3B, and 3C are intended to illustrate the various timing intervals that may be used in controlling ESS. The timing diagrams are not necessarily drawn to scale and the relative timing of ESS pulses between chambers during dual, bi- or multi-chamber applications may occur in any order that is expected to benefit the patient. For example, though the right ventricular and right atrial events and ESS pulses are shown to lead the left ventricular and left atrial events and ESS pulses in FIGS. 3B and 3C, in some patients the left chamber events and ESS pulses may lead the right chamber events and ESS pulses.

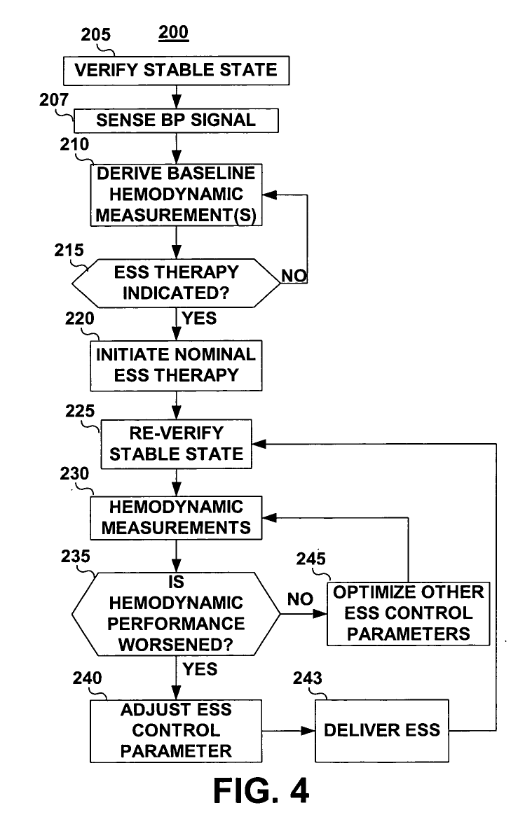

FIG. 4 is a flow chart summarizing a general method for regulating ESS based on hemodynamic monitoring. Initially, a baseline hemodynamic measurement will be performed when ESS is not enabled. At step 205, a stable state is verified to ensure hemodynamic measurements are reliable. Generally, verification of a stable state will include verifying normal sinus rhythm. In various embodiments, verification of a stable state may further include verification of other parameters such as, but not limited to: verifying a stable, sustained patient activity level, such as a resting activity level; verifying a stable, sustained patient posture, such as a prone position; or verifying a time of day, such as nighttime.

At step 207, a blood pressure signal is acquired for use in deriving one or more hemodynamic measures. The blood pressure signal may be obtained from a ventricle, such as the right ventricle as illustrated in FIG. 1. Alternatively or additionally, a blood pressure signal may be obtained from the left ventricle, the right or left atrium, or an arterial location. At step 210, one or more baseline hemodynamic measurements are derived using the sensed blood pressure signal. In one embodiment, a hemodynamic measurement is an estimated CO derived from a ventricular or arterial pressure signal using a pulse contour analysis. Pulse contour analysis generally refers to the analysis a pulse pressure waveform, typically an arterial pressure waveform, for estimating cardiac output. As used herein, however, “pulse contour analysis” refers to any analysis of a ventricular, atrial, or arterial pressure signal yielding any hemodynamic measurement derived there from. A method for estimating cardiac output based on a pulse contour analysis of the right ventricular pressure signal is generally disclosed in U.S. Pat. Appl. No. P11593, hereby incorporated herein by reference in its entirety. A method for estimating cardiac output based on an estimated flow contour derived from an arterial or ventricular pressure waveform is generally disclosed in U.S. Pat. Appl. No. P20222, hereby incorporated herein by reference in its entirety.

In another embodiment, the hemodynamic measurement includes an estimate of the mean pulmonary artery pressure (MPAP). MPAP may be estimated from the RVP signal according to methods generally disclosed in the above incorporated U.S. Pat. Appl. No. P11593 and in U.S. patent application Ser. No. 09/997,753, filed Nov. 30, 2001, also hereby incorporated herein by reference in its entirety

Other hemodynamic measurements that may be derived from a pressure signal include, but are not limited to, an estimated or measured end diastolic pressure, a stroke volume, a peak pressure, a peak rate of pressure change, a pulse pressure, or the like. For example, methods for deriving estimated pulmonary artery end diastolic pressure (ePAD) from a ventricular pressure signal are generally disclosed in U.S. Pat. No. 5,626,623 issued to Keival et al., and U.S. Pat. No. 6,580,946 B2 issued to Struble, both of which patents are hereby incorporated herein by reference in their entirety. It is recognized that one or more measurements may be obtained from the sensed pressure signal, which may be a ventricular, atrial or arterial signal. Measurements may be averaged over a selected interval of time, for example over several cardiac cycles, several seconds, or one or more minutes.

The baseline hemodynamic measurement(s) are evaluated at step 215 to determine if ESS therapy is indicated. Various criteria may be set by a clinician, and individualized for a particular patient need, for deciding when ESS should be initiated. In one embodiment, a threshold level for CO is defined. If CO falls below the threshold level, ESS is started at step 220 using nominally selected control parameters.

After initiating ESS therapy, hemodynamic monitoring is repeated to determine if ESS has had the intended beneficial effect, or at least not a detrimental effect on hemodynamic function. At step 225, re-verification of a stable state may be performed to ensure the hemodynamic measurements made after initiating ESS can be compared to the baseline measurements without confounding factors, such as a change in patient activity or cardiac rhythm. Re-verification of a stable state may include waiting a predefined interval of time to allow the hemodynamic response to ESS to reach a steady state.

At step 230, hemodynamic monitoring is repeated during ESS. As described above, one or more hemodynamic measurements are derived from at least a blood pressure signal. At step 235, the hemodynamic measurement(s) are evaluated to determine if hemodynamic function has worsened during ESS. In one embodiment, ESS is aimed at preventing a further decrease in CO. The benefit of ESS therapy in a heart failure patient, for example, may be to just maintain a resting level of CO without further decline in CO. If CO, estimated from the blood pressure signal, does not decrease during ESS as compared to the previously measured baseline CO, as determined at decision step 235, ESS therapy continues to be delivered at the nominal setting. Hemodynamic monitoring may continue, at step 230, on a continuous or periodic basis to detect any future decrease in CO and respond accordingly.

If hemodynamic performance has worsened during ESS, as determined at decision step 235, an ESS control parameter is adjusted at step 240. An ESS control parameter that is adjusted may be turning ESS off, adjusting a pacing rate, adjusting a pacing interval, adjusting an ESI, or adjusting the ESS ratio. After adjusting the ESS control parameter, ESS is delivered at step 243 according to the adjusted parameter, and hemodynamic measurements are repeated at step 230 after verifying a stable monitoring state (step 225). Once a maintained or improved hemodynamic performance is achieved, ESS is delivered according to the optimized control parameter. Other ESS control parameters may be optimized at step 245 in an attempt to further improve hemodynamic performance.

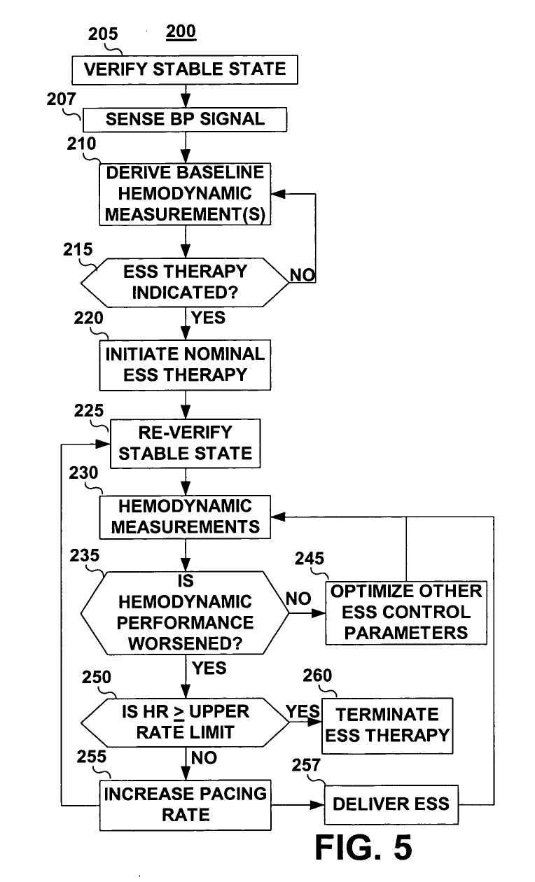

FIG. 5 is a flow chart summarizing one embodiment for regulating ESS that includes adjusting a pacing rate in response to hemodynamic monitoring. In FIG. 5, steps 205 through 235 correspond to identically numbered steps shown in FIG. 4. If a worsened hemodynamic performance is determined at decision step 235, based on a pressure signal-derived hemodynamic parameter such as CO, the current heart rate is compared to an upper rate limit at step 250. The current rate may be a paced or intrinsic rate. If the HR is less than the HR limit, a pacing rate is increased by a predetermined increment at step 255. In the example of the dual chamber ESS application shown in FIG. 3, if the HR is less than the upper rate limit, the atrial pacing rate is adjusted to an increment above the HR. Since one effect of ESS is a slowing of the intrinsic HR, CO can decrease in response to ESS. As such, if a decrease in CO is measured after enabling ESS, one response to the decreased CO is to increase the heart rate by pacing.

After increasing the pacing rate at step 255, ESS is delivered according to the new control parameter at step 257, and hemodynamic monitoring continues at step 230 after verifying stable monitoring conditions at step 225. If the estimated CO or other hemodynamic measurements still indicate a worsened hemodynamic performance, the pacing rate may be incrementally increased up to a predefined maximum HR limit. If the maximum HR limit is reached, as determined at step 250, and CO is still worse than the baseline measure, ESS is terminated at step 250.

ESS may be terminated abruptly or terminated through a weaning process. An abrupt termination of ESS may cause a sudden, undesirable, hemodynamic perturbation. As such, ESS termination may involve progressively adjusting ESS control parameters to gradually remove any potentiation effect over an interval of time. A weaning process may involve, for example, progressively decreasing the ESS ratio (increasing the number of cardiac cycles between each ESS pulse). The weaning process may alternatively or additionally involve progressively increasing an ESI, for example the ventricular ESI. As ESI is increased, the potentiation effect declines thereby weaning the heart from the effects of ESS.

If the pacing rate adjustment results in a maintained or improved hemodynamic performance, as determined at decision step 235, optional optimization of other ESS control parameters may be performed at step 245.

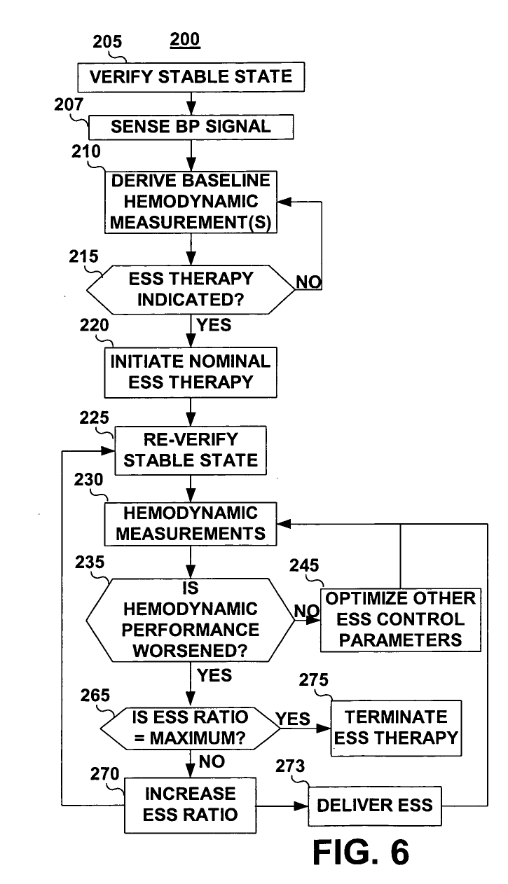

FIG. 6 is a flow chart summarizing one embodiment of a method for regulating ESS that includes adjusting the ESS ratio in response to a hemodynamic measure. In FIG. 6, steps 205 through 235 correspond to identically numbered steps shown in FIG. 4. If a worsened hemodynamic performance is determined at decision step 235, based on a pressure signal-derived hemodynamic parameter such as CO, the current ESS ratio (HR to ESS rate) is compared to a predefined maximum ratio at step 265. If the ESS ratio is less than the maximum, the ESS ratio is increased at step 270, i.e., the number of sensed or paced events between each ESS pulse is increased by one. ESS is delivered at step 273 at the adjusted ESS ratio. By increasing the ESS ratio, the effect of ESS on the heart rate may be reduced. The potentiation effect of ESS can persist for several cardiac cycles. As such the ESS ratio may be increased in order to cause the intrinsic heart rate to rise without losing the potentiation effect on post extra systolic cardiac cycles.

If the ESS ratio reaches a maximum and the hemodynamic performance is not at least maintained or improved compared to baseline measurements, ESS therapy is terminated at step 275, either abruptly or through a weaning process as described previously. If an ESS ratio is found that results in maintained or improved hemodynamic performance, optional optimization of other ESS control parameters is performed at step 245. Continued monitoring of hemodynamic measurements is performed on a continuous or periodic basis at step 230 to detect any decline in hemodynamic performance requiring further adjustment of ESS control parameters.

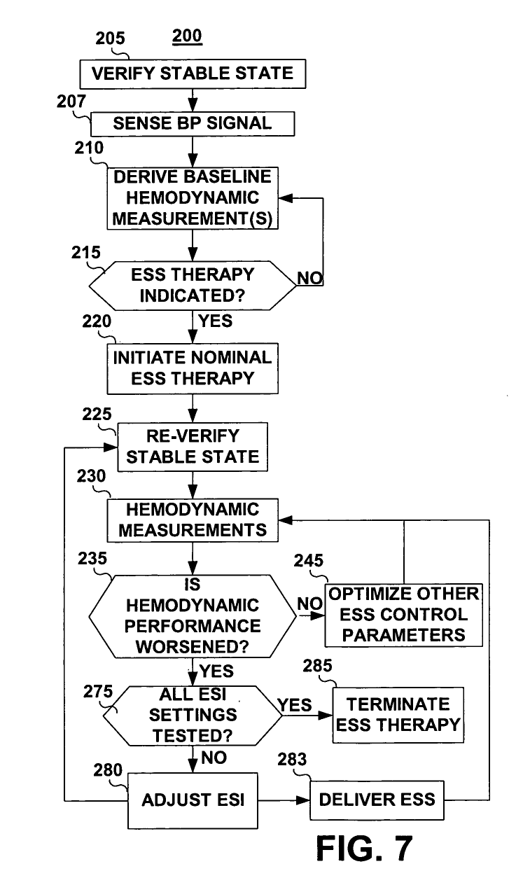

FIG. 7 is a flow chart summarizing another embodiment of a method for regulating ESS that includes adjusting an ESI in response to a hemodynamic measure. Steps 205 through 235 correspond to identically numbered steps shown in FIG. 4. If a worsened hemodynamic performance is determined at decision step 235, based on a pressure signal-derived hemodynamic parameter such as CO, an ESI may be adjusted. In the dual chamber application illustrated in FIG. 3A, the AESI may be adjusted, potentially reducing the potentiation effect in the atrium. Alternatively or additionally, the VESI may be adjusted, potentially reducing the potentiation effect in the ventricle. The reduced potentiation effect in the atrium and/or ventricle may act to increase the heart rate, resulting in a net increase in CO. In some cases, an increased potentiation effect may occur after adjusting an ESI. The increased potentiation effect may also have a net positive effect on hemodynamic performance. In some embodiments, the timing of the atrial ESS pulse may be controlled based on an AV ESI as shown in FIG. 3A. As such, the AV ESI may be adjusted in response to a worsened measurement of hemodynamic performance resulting in a change in the potentiation effect and net result on CO.

An ESI is adjusted at step 280 to a setting within a predetermined minimum and maximum ESI range. ESS is delivered at step 283 according to the adjusted ESI. Hemodynamic measurements are repeated until all ESI settings have been tested, as determined at decision step 275, or until hemodynamic performance is determined to be maintained or improved relative to baseline hemodynamic measurements (decision step 235). If adjustment of an ESI setting does not result in maintained or improved hemodynamic performance, ESS is terminated at step 285, either abruptly or through a weaning process.

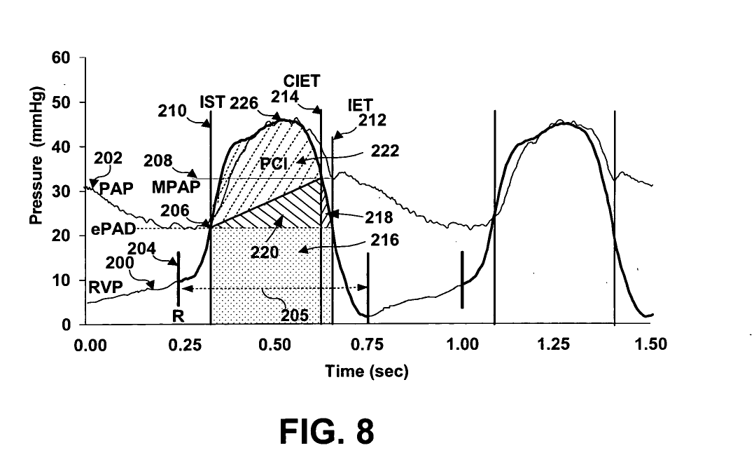

FIG. 8 shows a right ventricular pressure (RVP) waveform and a pulmonary artery pressure (PAP) waveform and illustrates a number of hemodynamic measures that may be derived from a pressure signal for use in regulating ESS. The RVP signal 200 is obtained from a pressure sensor implanted in the right ventricle, and the PAP signal 202 is obtained from a pressure sensor implanted in the pulmonary artery. The RVP and PAP pressure signals obtained during ESS may be altered compared to those shown in FIG. 8, due to the extra systolic stimulus, which may evoke a weak mechanical response, and a potentiation effect post extra systolic beats. However, the general principles for deriving a hemodynamic parameter from a pressure signal may still be applied. Generally, a hemodynamic parameter can be derived by identifying a fiducial point on the pressure signal and/or using fiducial points for defining areas under the pressure curves for estimating stroke volume and calculating an estimated cardiac output there from.

The RVP signal 200, for example, can be used to estimate pulmonary artery end diastolic pressure (ePAD) 206, mean pulmonary artery pressure (MPAP) 208, and CO based on a pulse contour integral (PCI) 222. For a detailed description of methods for estimating CO based on pulse contour analysis, reference is made to the above-incorporated U.S. Pat. Appl. No. P11593. Briefly, the RVP signal is acquired during a sensing window 205 following an R-wave event 204. The ePAD 206 is derived as the RVP at the time of the maximum dP/dt of the RVP signal. This time point is considered an estimate of the start of ejection time and may be used to define an integration start time (IST) 210. An integration end time (IET) 212 corresponds to the time the falling RVP signal crosses ePAD 206. The area under the RVP signal 200 between the IST 210 and IET 212 can be used to estimate stroke volume.

The estimate of stroke volume can be improved by correcting the area under the RVP signal between the IST 210 and IET 212. For example, the area 216 under ePAD can be subtracted from the integrated area since this area is more likely associated with rise in RVP during the pre-ejection phase. A corrected integration end time (CIET) 214 can be determined as the time that the RVP signal magnitude equals an estimated MPAP. MPAP can be estimated as a weighted average of the peak RVP 226 and ePAD 206. Weighting factors can be determined from the systolic and diastolic time intervals measured during the cardiac cycle. Using the time that the falling RVP signal 200 equals the estimated MPAP 208 as a CIET 214, an area 218 is removed from the pulse contour area used for estimating stroke volume. Another area 220 can be estimated from the computed MPAP 208, ePAD 206, and IST 210 and CIET 214. The remaining pulse contour integral (PCI) 222 may be used as an estimate of stroke volume. When the PAP signal 202 is available, PA end diastolic pressure and MPAP can be measured directly.

In another embodiment, fiducial points may be identified from an arterial pressure signal, such as PAP signal 202, or a ventricular pressure signal, such as RVP signal 200, for estimating a flow contour as generally disclosed in the above-incorporated U.S. Pat. Appl. No. P20222. From the estimated flow contour, an estimated stroke volume can be computed and, knowing the heart rate, an estimated CO can be computed.

It is recognized that the hemodynamic parameters derived from a pressure waveform may vary between embodiments as well as the methods used to derive such parameters. Furthermore, methods such as the pulse contour analysis applied to a RVP signal or the flow contour estimation method applied to an arterial or ventricular pressure signal may be modified to account for changes in the pressure signal contour due to ESS. Derived hemodynamic parameters may be determined in physical units after calibration procedures. However, relative changes in a non-calibrated hemodynamic parameter can generally be used effectively in regulating ESS.

Thus, a method and apparatus for controlling ESS using hemodynamic parameters derived from a pressure signal have been presented in the foregoing description with reference to specific embodiments. It is appreciated that various modifications to the referenced embodiments may be made without departing from the scope of the invention as set forth in the following claims.

1. A method, comprising:
sensing a blood pressure signal;
deriving a hemodynamic measure using the sensed blood pressure signal,
adjusting an extra systolic stimulation control parameter in response to the hemodynamic measure, and
delivering extra systolic stimulation pulses according to the adjusted control parameter.

2. The method of claim 1, wherein sensing the blood pressure signal includes sensing a ventricular blood pressure signal.

3. The method of claim 1 wherein deriving the hemodynamic measure includes deriving an estimated cardiac output using the sensed blood pressure signal.

4. The method of claim 3 wherein deriving the estimated cardiac output includes computing a pulse contour integral using the blood pressure signal

5. The method of claim 1 wherein deriving the hemodynamic measure includes estimating a flow contour using the blood pressure signal.

6. The method of claim 1 wherein deriving the hemodynamic measure includes deriving an estimated end diastolic pressure.

7. The method of claim 1 wherein deriving the hemodynamic measure includes deriving an estimated mean arterial pressure.

8. The method of claim 1 wherein adjusting the extra systolic stimulation control parameter includes adjusting a parameter for enabling or terminating the delivery of extra systolic stimulation pulses.

9. The method of claim 1 wherein adjusting the extra systolic stimulation control parameter includes adjusting any of: a pacing rate, a pacing interval, an extra systolic stimulation interval, and an extra systolic stimulation ratio.

10. The method of claim 1 wherein sensing the blood pressure signal includes sensing an arterial blood pressure signal.

11. A medical device, comprising:
a blood pressure sensor for sensing a blood pressure signal;
a signal processing module for deriving a hemodynamic measure from the sensed blood pressure signal;
a therapy delivery module for delivering extra systolic stimulation pulses;
a timing and control module for controlling the delivery of extra systolic stimulation pulses delivered by the therapy delivery module in response to the derived hemodynamic measure.

12. The medical device of claim 11 wherein the blood pressure sensor is adapted for deployment in the right ventricle.

13. The medical device of claim 11 wherein the blood pressure sensor is adapted for deployment in the pulmonary artery.

14. The medical device of claim 11 wherein the hemodynamic measure is an estimated cardiac output.

15. A medical device, comprising:
means for obtaining a blood pressure signal;
means for deriving a hemodynamic measure using the blood pressure signal;
means for adjusting an extra systolic stimulation control parameter in response to the derived hemodynamic measure;
means for delivering extra systolic stimulation pulses according to the extra systolic stimulation control parameter.

16. The device of claim 15, wherein the means for obtaining a blood pressure signal includes means for sensing a ventricular pressure.

17. The device of claim 15, wherein the means for obtaining a blood pressure signal includes means for sensing an arterial pressure.

18. The device of claim 15, wherein the means for deriving a hemodynamic measure includes means for computing a pulse contour integral using the blood pressure signal.

19. The device of claim 15 wherein the means for deriving a hemodynamic measure includes means for estimating a mean arterial pressure.

20. The device of claim 15 wherein the means for deriving a hemodynamic measure includes means for estimating an end diastolic pressure.

21. The device of claim 15 wherein the means for adjusting an extra systolic stimulation control parameter includes means for adjusting any of: a pacing rate, an extra systolic stimulation ratio, a pacing interval, and an extra systolic interval.

22. The device of claim 15 wherein the means for adjusting an extra systolic stimulation control parameter includes means for enabling or disabling the means for delivering extra systolic stimulation pulses.

23. The device of claim 15 wherein the means for delivering extra systolic stimulation pulses includes means for weaning extra systolic stimulation pulses.

24. The device of claim 23 wherein the means for weaning extra systolic stimulation pulses includes means for progressively increasing any of: an extra systolic stimulation ratio and an extra systolic stimulation interval.

25. A computer readable medium for storing a set of instructions which when implemented in a system cause the system to:
acquire a blood pressure signal;
derive a hemodynamic measure using the blood pressure signal;
adjust an extra systolic stimulation control parameter in response to the derived hemodynamic measure; and
deliver extra systolic stimulation pulses according to the extra systolic stimulation control parameter.

26. The computer readable medium of claim 25 wherein the blood pressure signal is a ventricular blood pressure signal.

27. The computer readable medium of claim 25 wherein the blood pressure signal is an arterial signal.

28. The computer readable medium of claim 25 wherein the derived hemodynamic measure is an estimated cardiac output.

29. The computer readable medium of claim 25 wherein deriving a hemodynamic measure includes performing a pulse contour analysis of the blood pressure signal.

30. The computer readable medium of claim 25 wherein deriving a hemodynamic measure includes performing a flow contour estimation using the blood pressure signal.

31. The computer readable medium of claim 25 wherein the extra systolic stimulation control parameter is any of: a pacing rate, an extra systolic stimulation ratio, a pacing interval, and an extra systolic stimulation interval.

32. The computer readable medium of claim 25 wherein the extra systolic stimulation control parameter is a parameter that enables or terminates extra systolic stimulation pulses.

33. The computer readable medium of claim 32 further including instructions which cause the system to perform a weaning procedure when extra systolic stimulation is terminated.

34. The computer readable medium of claim 33 wherein the weaning procedure includes progressively adjusting any of: an extra systolic stimulation ratio, and an extra systolic stimulation interval.
